(19) United States
(12) Patent Application Publication
(10) Pub. No.: US 2008/0299537 A1
(43) Pub. Date: Dec. 4, 2008

(54) Apparatus and Methods For Selecting Capacitated Spermatozoa and Uses Thereof

(75) Inventors: Michael Eisenbach, Rehovot (IL); Anat Bahat, Rehovot (IL)

Correspondence Address: BROWDY AND NEIMARK, P.L.L.C.;624 NINTH STREET, NW, SUITE 300, WASHINGTON, DC, 20001-5303, US

(73) Assignee: YEDA RESEARCH AND DEVELOPMENT, Rehovot (IL)

(21) Appl. No.: 10/573,412

(22) PCT Filed: Sep. 25, 2003
(86) PCT No.: PCT/IL2003/000768

§ 371 Date: Aug. 18, 2008

(51) Int. Cl. A01N 1/02; C12M 1/34; G01N 33/53

(52) U.S. Cl. 435/2; 435/286.1; 435/7.2

(57) ABSTRACT

The present invention relates to apparatus, systems and methods for generating a subpopulation of spermatozoa enriched for capacitated spermatozoa, by exposure of a population of spermatozoa to a suitable temperature gradient, and retrieving the enriched subpopulation of spermatozoa for further applications, such as, for diagnosis or fertility treatments.

### FIELD OF THE INVENTION

The present invention relates to apparatus, systems and methods for generating a population of spermatozoa enriched with capacitated spermatozoa, using a suitable temperature gradient. The invention further provides methods for diagnosis and improving the outcome of fertility treatments by using a population of spermatozoa enriched with capacitated spermatozoa.

### BACKGROUND OF THE INVENTION

In mammals, only a small fraction of the spermatozoa ejaculated directly into the female genital tract (about 50-8,000 millions) reaches the oviductal isthmus, and much lower numbers (about 250 in rabbits and humans) reach the fertilization site (isthmic-ampullary junction in rabbits and ampulla in humans) after ovulation. Upon entering the oviductal isthmus, spermatozoa become trapped and form a reservoir. Only spermatozoa that become capacitated, namely spermatozoa that acquire a state of readiness for fertilizing the egg, are released, a few at a time, from the reservoir. Without a guidance mechanism, such as chemotaxis, there is a low probability that these few spermatozoa will reach the egg. However, peristaltic movements of the oviduct may prevent the formation of a long-range chemoattractant gradient and restrict chemotaxis to a short distance from the egg.

Another potential cue for guiding spermatozoa to the site of fertilization is the temperature difference that exists between the cooler isthmus and the warmer fertilization site at ovulation. A temperature difference of approximately 2° C. on average was found during ovulation between the isthmus and the isthmic-ampullary junction in rabbits, and about 0.7° C. between the isthmus and the ampulla in mated pigs.

Thermotaxis is defined as an oriented movement in a temperature gradient. Human spermatozoa can sense and respond by thermotaxis to a small temperature difference, of 2° C., between 37° C. and 39° C. Furthermore, thermotactic responsiveness is acquired during sperm capacitation, as is chemotactic responsiveness (Cohen-Dayag, A., et al., Proc. Natl. Acad. Sci. U.S.A., 1995. 92: 11039-43). Thermotaxis of mammalian spermatozoa is disclosed in Bahat et al., Nature Med., 9:149-50, 2003.

Methods for assaying the quality of spermatozoa on the basis of biochemical markers or functional criteria, including motility and binding to components of the ovum or zona pellucida, are known in the art.

U.S. Pat. No. 6,558,911 discloses a method for assaying fertility in an animal comprising measurements of surface ubiquitination of sperm in a semen sample and correlating the measured surface ubiquitination with fertility, where increased levels of ubiquitination are indicative of decreased rates of fertility.

U.S. Pat. No. 6,541,206 discloses a method of testing sperm quality comprising detecting and measuring amount of testis-specific chaperone protein in a sperm sample by a chaperone protein-specific immuno-assay, where the testis-specific chaperone protein is an HspA2 testis-specific chaperone protein. An increased amount of the chaperone protein per given amount of sperm in the sample indicates a high sperm quality.

U.S. Pat. No. 6,465,197 discloses a method for assaying mammalian sperm in a sperm sample for reproductive competence comprising exposing a portion of the sperm sample to disulfide bond reducing conditions following culture with a cell-free oocyte extract under conditions favoring the formation of microtubule structures in the culture. The method further comprises visualizing the microtubule formation in the culture where the formation of a sperm aster at the base of the sperm head determines the reproductive competence of the sperm.

U.S. Pat. No. 5,219,729 discloses a diagnostic assay for predicting sperm fertilizing potential which comprises contacting fragments of mammalian zona pellucida of the same oocyte having functionally equivalent sperm binding activity with sperm from the same species under binding conditions, and with sperm from the same species of known fertilizing potential under binding conditions, and comparing the resulting sperm binding.

There is an unmet need for evaluation and selection criteria for spermatozoa in mammals and for enriching populations of spermatozoa with capacitated spermatozoa, using assays and methods that do not impair sperm viability. This need is especially acute for diagnosis and for enhancing sperm quality in the course of fertility treatments for improving the outcome of such treatments.

### SUMMARY OF TIE INVENTION

It is an object of the present invention to provide apparatus, systems and methods for obtaining a population of spermatozoa enriched with capacitated spermatozoa. The present invention relates to the enrichment of a population of spermatozoa with capacitated spermatozoa by subjecting the population to a suitable temperature gradient. The present invention further relates to the thermotactic responsiveness of the capacitated cells and applications and uses thereof.

The apparatus, systems and methods of the present invention are advantageous over methods known in the art for diagnosis and selection of capacitated spermatozoa, as the present invention provides non-toxic assays, which enable enrichment of a sperm population with capacitated spermatozoa while maintaining sperm motility. Thus, the enriched sperm population obtained according to the present invention may be used, without further manipulations, for any desired application, and particularly for fertility treatments.

In addition, applying the apparatus, systems and methods of the present invention provides a sperm population enriched with capacitated spermatozoa exhibiting thermotactic responsiveness. Thus, the improvement in sperm quality according to the present invention provides an enriched sperm subpopulation with an advantageous functionality, which is particularly beneficial for improving the success of fertilization.

According to a first aspect the present invention discloses an apparatus suitable for generating a subpopulation of spermatozoa enriched for capacitated spermatozoa and for selection of the enriched subpopulation. The apparatus comprises at least two compartments adapted for the maintenance of viable and motile spermatozoa and means for the generation of a temperature gradient between the at least two compartments. The apparatus may further comprise means for monitoring cellular movement between the culture compartments.

According to one embodiment, the present invention provides an apparatus for selecting a subpopulation of spermatozoa, comprising:
(a) a culture chamber having at least one first compartment and at least one second compartment and a passage enabling access of spermatozoa between the at least one first compartment to the at least one second compartment; and,
(b) means for generating a temperature gradient between the at least one first compartment and the at least one second compartment such that the temperature in said at least one first compartment is lower than the temperature in said at least one second compartment.

According to another embodiment, the passage between said at least one first compartment and said at least one second compartment comprises a discrete pathway, typically of dimensions smaller than those of the compartments on either end.

According to yet another embodiment, the culture chamber is adapted for containing culture medium suitable for maintaining the motility of mammalian spermatozoa or human spermatozoa. According to yet another embodiment, the culture chamber is sterile or aseptic.

According to yet another embodiment, the culture chamber is further adapted for semen washing. According to yet another embodiment, the procedure of semen washing is selected from the group consisting of: swim up, discontinuous (density) gradient and simple (centrifuge) wash.

According to yet another embodiment, the culture chamber comprises a biocompatible material. In yet another embodiment, the culture chamber comprises a material selected from the group consisting of: glass, polycarbonate, polyethylene, polyurethane, ethylene-vinylacetate copolymer and polyolefins.

According to yet another embodiment, the temperature gradient is discrete or continuous, wherein the temperatures within the temperature gradient are suitable for maintaining sperm viability. According to yet another embodiment, the difference between the highest and the lowest temperatures of the temperature gradient is no more than 20° C. and at least 0.05° C.

According to yet another embodiment, the passage further comprising a matrix between the at least one first compartment and the at least one second compartment, optionally, the matrix is at least partly permeable to spermatozoa. Preferably the matrix is permeable to capacitated spermatozoa.

According to yet another embodiment, the matrix comprises a material selected from the group consisting of: a biocompatible gel, fibrin substrate, silicon, carbon blocks or fibers, polysaccharides and collagen.

According to yet another embodiment, the apparatus further comprising means for monitoring sperm motility. According to yet another embodiment, the culture chamber is disposable.

It is to be understood that the apparatus according to the present invention is not limited to any design, size, shape or geometry. Any apparatus, which can provide an improved spermatozoa subpopulation in accordance to the principles of the present invention, particularly a sperm population enriched with capacitated spermatozoa, may be used.

According to a second aspect the present invention provides a system for enriching a sample of spermatozoa with capacitated spermatozoa and for retrieving the enriched spermatozoa for further applications. The system of the invention comprises exposing a population of spermatozoa to a suitable temperature gradient in a device having means for generating a temperature gradient and for retrieving the enriched sperm population.

According to one embodiment, the present invention provides a system for generating a subpopulation of spermatozoa enriched for capacitated spermatozoa, comprising:
(a) a culture chamber having at least one first compartment adapted for holding viable spermatozoa in a culture medium and at least one second compartment containing a culture medium suitable for maintaining viable spermatozoa and a passage enabling spermatozoa access between the at least one first compartment and the at least one second compartment;
(b) means for generating a temperature gradient in the culture chamber between the at least one first compartment and the at least one second compartment, such that the temperature in said at least one first compartment is lower than the temperature in said at least one second compartment; and, optionally
(c) means for retrieving spermatozoa from the at least one second compartment.

According to yet another embodiment, the culture medium of the system is suitable for maintaining viable mammalian spermatozoa, optionally, human spermatozoa. According to yet another embodiment, the culture chamber comprises a biocompatible material. The material may be selected from the group consisting of: glass, polycarbonate, polyethylene, polyurethane, ethylene-vinylacetate copolymer and polyolefins.

According to yet another embodiment, the culture chamber is sterile or aseptic and according to yet another embodiment, the culture chamber is disposable.

According to yet another embodiment, the temperature gradient between the at least one first compartment to the at least one second compartment of the system is discrete or continuous.

According to yet another embodiment, the passage of the system comprises a matrix between the at least one first compartment and the at least one second compartment. According to yet another embodiment, the matrix is selectively permeable to spermatozoa According to yet another embodiment, the permeable matrix is selected from the group consisting of: a biocompatible gel, collagen, fibrin substrate, carbon blocks or fibers, polysaccharides and silicon.

According to yet another embodiment, the system further comprising means for monitoring sperm motility.

According to yet another embodiment, the temperatures within the temperature gradient of the system are suitable for maintaining sperm viability, wherein the difference between the highest and the lowest temperatures of the temperature gradient is between 0.05° C. to 20° C.

The present invention discloses the unexpected discovery that spermatozoa respond thermotactically to very low temperature gradients, as low as 0.5° C. or less, whereas the magnitude of the thermotactic response obtained at low temperature gradients and at higher temperature gradients, of about 2° C., is similar.

According to yet another embodiment, a subpopulation of spermatozoa enriched with capacitated spermatozoa accumulates in the at least one second compartment upon introduction of a sperm population into the at least one first compartment of the culture chamber of the system and exposure of the sperm population to a temperature gradient generated between the first compartment and the at least one second compartment.

According to yet another embodiment, the system of the invention is suitable for washing semen and obtaining a population of spermatozoa which is essentially devoid of secondary components, such as, cell debris, white blood cells and prostaglandins. Particularly, the system of the invention is suitable for combining methods for semen washing with methods for generating a subpopulation of spermatozoa enriched for capacitated spermatozoa.

According to a third aspect the present invention provides methods and assays for evaluating the quality of spermatozoa and for enriching for a subpopulation of capacitated spermatozoa. The methods of the present invention relate to the generation of a spermatozoa subpopulation enriched with capacitated spermatozoa and can be used in the course of fertility treatments, optionally in combination with other methods for improving semen quality, such as, semen washing methods, for improving the outcome of such treatments. The assays further include the step of evaluating the thermotactic response of a population of spermatozoa with respect to the thermotactic response of standard.

According to one embodiment, the present invention provides a method for generating a subpopulation of spermatozoa enriched for capacitated spermatozoa, comprising:
(a) providing a population of spermatozoa in at least one first site;
(b) exposing the population of (a) to a temperature gradient induced between the at least one first site and at least one second site, wherein the temperature at the at least one first site is lower than the temperature at the at least one second site;
(c) obtaining a subpopulation of spermatozoa enriched with capacitated spermatozoa from the at least one second site; and, optionally,
(d) repeating step (b) at least once, with the population obtained in (c).

According to another embodiment, step (b) of the method of the invention further comprises monitoring of sperm motility from the at least one first site to the at least one second site. According to yet another embodiment, sperm motility is evaluated in comparison to a standard.

According to yet another embodiment, the method further comprises semen washing prior to step (a). According to yet another embodiment, the semen washing procedure is selected from the group consisting of: swim-up, discontinuous (density) gradient and simple (centrifuge) wash.

According to yet another embodiment, the population of spermatozoa provided in the method of the invention comprises non-human mammalian spermatozoa or human spermatozoa.

According to yet another embodiment, the temperature gradient of the method of the invention is discrete or continuous, wherein the temperatures within the temperature gradient are suitable for maintaining sperm viability. The difference between the highest and the lowest temperatures of the temperature gradient is between 0.05° C. to 20° C.

According to yet another embodiment, the method of the invention further comprises retrieving a population of spermatozoa after step (b) from the at least one second site. According to yet another embodiment the retrieved population is used for diagnosis or used for a fertility treatment. According to yet another embodiment, the fertility treatment is selected from the group consisting of: artificial insemination, intrauterine insemination (IUI), intracytoplasmic sperm injection (ICSI), in vitro fertilization (IVF), micromanipulation IVF and intra-vaginal fertilization.

According to one embodiment, the present invention provides an assay for evaluating sperm quality in a population of spermatozoa, comprising:
(a) providing a population of spermatozoa in a first site;
(b) exposing the population of (a) to a temperature gradient induced between the first site and at least one second site, such that the temperature at the at least one second site is higher than at said first site; and,
(c) evaluating the percentage of spermatozoa within the population accumulated at the second site of (b) in comparison to a standard sperm population, wherein the percentage of spermatozoa migrating along the temperature gradient between said first site and the at least one second site is a measure of sperm quality.

According to another embodiment, the population of spermatozoa provided in the assay of the invention comprises non-human mammalian spermatozoa or human spermatozoa.

According to yet another embodiment, the temperature gradient in the assay of the invention is discrete or continuous, wherein the temperatures within the temperature gradient are suitable for maintaining sperm viability. The difference between the highest and the lowest temperatures of the temperature gradient is between 0.05° C. to 20° C.

### BRIEF DESCRIPTION OF THE DRAWINGS

FIG. 1 is a scheme of a modified Zigmond Chamber comprising a first compartment (1), a second compartment (2), a temperature-insulating partition wall (3) two thermistors (4) and two tubes (5) containing water from two distinct water baths (6, 7) having different temperatures.

FIG. 2 is a scheme of a culture chamber comprising a first compartment (1), a plurality of second compartments (2) and a plurality of passages (8) each passage extending between the first compartment and one of the second compartments.

FIG. 3A is a scheme of a culture chamber suitable for applying a temperature gradient to a sample of spermatozoa selected by a swim up method, comprising a first compartment (1) having a bottom part (9) and a top part (10), a second compartment (2) and a passage (8) between the first and the second compartments.

FIGS. 3B-E are schemes of culture chambers suitable for applying a temperature gradient to a sample of spermatozoa, each culture chamber comprises at least one first compartment (1), a second compartment (2) and a passage (8) extending between the at least one first compartment and the second compartment.

FIG. 4 is a longitudinal cross-section (A) and a top cross-section (B) of a system comprising a culture chamber (11) having a first compartment with temperature T₁ (1), a second compartment with temperature T₂ (2), where T₁<T₂, a coverslip (12) covering the two compartments and the bridge (13), spermatozoa that swam up from the wells to the top of the bridge (14) and means (15) for monitoring movement of the cells on top of the bridge according to kinetic parameters (C).

FIG. 5 demonstrates the thermotactic response of capacitated rabbit spermatozoa moving along two wells having the same temperature (37° C./37° C., I; 39° C./39° C., II) or different temperatures (37° C./39° C., III).

FIG. 6 demonstrates the thermotactic response of capacitated rabbit spermatozoa moving along two wells having the same temperature (37° C./37° C., I) or different temperatures (37° C./37.5° C., II; 37° C./38° C., III; 37° C./39° C., IV).

FIG. 7 demonstrates the thermotactic response of non-capacitated and capacitated rabbit spermatozoa across two wells having the same temperature (37° C./37° C., I; 39° C./39° C., II) or different temperatures (37° C./39° C., III).

FIG. 8 shows a fluorescence-microscope picture of A23187-induced rabbit spermatozoa after 15 h incubation in capacitating conditions.

FIG. 9 presents thermotactic response of human spermatozoa across two wells having the same temperature (37° C./37° C., I; 39° C./39° C., II) or different temperatures (37° C./39° C., III).

FIG. 10 shows thermotactic response of human spermatozoa, incubated with a BWW medium (I-III), across two wells having the same temperature (37° C./37° C., I; 39° C./39° C., II) or a temperature gradient (37° C./39° C., III) vs. non-capacitated human spermatozoa that were exposed to a temperature gradient (37° C./39° C., IV).

### DETAILED DESCRIPTION OF THE INVENTION

The present invention discloses apparatus, systems and methods suitable for enriching a subpopulation of spermatozoa for capacitated spermatozoa. Capacitation refers to the ability of spermatozoa to adhere to, penetrate and fertilize susceptible ova. Mammalian spermatozoa must undergo alterations in the plasma membrane in order to acquire fertilizing capability. The process during which the spermatozoa undergo these alterations in their membrane is termed capacitation and occurs naturally in the female reproductive tract once the semen has been deposited. Penetration and fertilization not only require potentiality of the spermatozoa to achieve a functional status, but also require that favorable conditions exist in the oviduct environment. If favorable conditions exist in the mammalian oviduct, spermatozoa become capacitated and penetrate the ova. Thereafter, fertilization ensues and embryonic development begins.

The present invention discloses an apparatus suitable for exposing a population of spermatozoa to a temperature gradient. According to one embodiment, the present invention provides an apparatus for selecting a subpopulation of spermatozoa, comprising:
(a) a culture chamber having at least one first compartment, at least one second compartment and at least one passage between the at least one first compartment and the at least one second compartment; and,
(b) means for generating a temperature gradient between said at least one first compartment and said at least one second compartment such that the temperature in the at least one first compartment is lower than the temperature in the at least one second compartment.

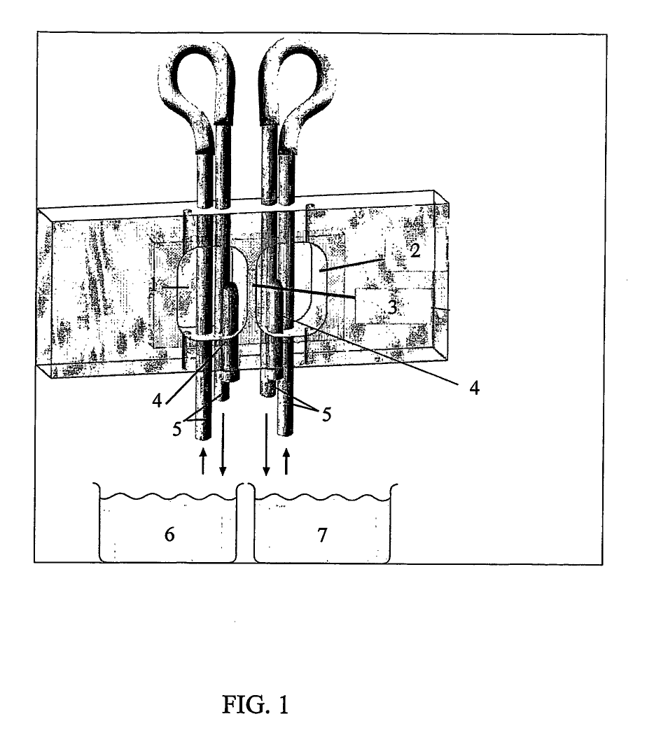

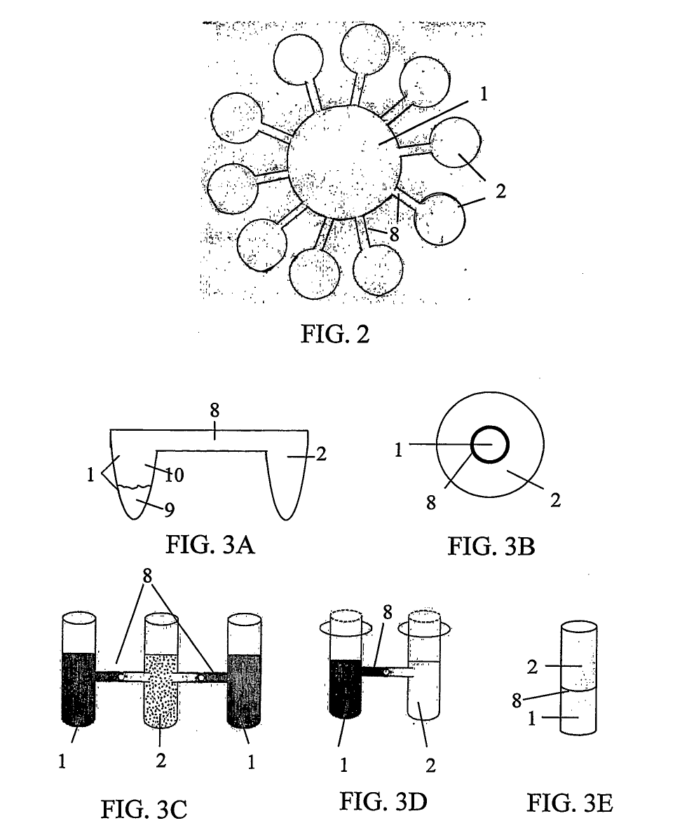

The apparatus according to the present invention is not limited to any design, size, shape or geometry. Any apparatus which can provide an improved spermatozoa subpopulation in accordance to the principles of the present invention, particularly a sperm population enriched with capacitated spermatozoa, may be used, including the designs represented in FIGS. 1, 2 and 3A-E and the fertilization and culture container disclosed in U.S. Pat. No. 6,050,935.

According to yet another embodiment, the present invention provides a system for enriching a subpopulation of spermatozoa for capacitated spermatozoa, comprising:
(a) a culture chamber having at least one first compartment and at least one second compartment each compartment adapted for containing a culture medium and adapted for holding viable spermatozoa, and a passageway between the first and second compartments enabling access of the spermatozoa; and
(b) means for generating a temperature gradient in the culture chamber between the at least one first compartment and the at least one second compartment, such that the temperature in said at least one first compartment is lower than the temperature in said at least one second compartment; and, optionally
(c) means for retrieving spermatozoa from the at least one second compartment.

The apparatus and system according to the present invention comprise a culture chamber adapted for maintaining the motility of mammalian spermatozoa or of human spermatozoa According to one embodiment, the culture chamber is sterile or aseptic. In addition, the culture chamber may be disposable. The culture chamber may comprise any biocompatible material known in the art, preferably of a tissue culture (TC) grade, wherein the biocompatible material does not impair sperm quality, such that semen or spermatozoa can be maintain therein without damage and preferably without adhering thereto. Examples of suitable materials include glass, polycarbonate, polyethylene, polyurethane, ethylene-vinylacetate copolymer and polyolefins among others.

The apparatus according to the present invention further comprises means for generating a temperature gradient between the at least one first compartment and the at least one second compartment such that the temperature in the at least one first compartment is lower than the temperature in the at least one second compartment.

The temperatures included within the temperature gradient must be suitable for maintaining sperm viability. According to certain embodiments the temperature range spanned between the highest and the lowest temperatures of the temperature gradient can be as small as 0.05° C., though typically it is at least a few tenths of a degree, e.g. about 0.5° C. The temperature range spanned between the highest and the lowest temperatures of the temperature gradient may be as large as 20° C., though typically is will not exceed a few degrees.

According to one embodiment, the highest and lowest temperatures of the temperature gradient are each preferably within 5° C. of normal body temperature, for example, the highest temperature of a temperature gradient is 39° C. and the lowest temperature of the temperature gradient is 30° C. Generally, the lowest temperature of the temperature gradient is not lower than 20° C. and the highest temperature of the temperature gradient is not higher than 40° C. In addition, the temperature gradient may be either discrete or continuous.

The culture chamber of the apparatus and system of the present invention further comprises at least one passage between the at least one first compartment and the at least one second compartment, wherein the at least one passage enables the access of spermatozoa from one compartment to the other compartment. The passage may further comprise a matrix between the at least one first compartment and the at least one second compartment, optionally, the matrix is at least partly permeable to spermatozoa. The matrix may comprise any biocompatible material known in the art, optionally of a tissue culture (TC) grade, wherein the biocompatible material does not appreciably impair sperm quality, such that semen or spermatozoa can pass therethrough without damage and without adhering thereto. The matrix may comprise a biocompatible gel of collagen, fibrin substrate, silicon and carbon blocks or fibers among others.

The culture chamber of the apparatus and system of the present invention may be adapted for semen washing and may be used for applying the methods of the present invention in combination with semen washing. Procedure for improving semen quality by washing a sample of semen from unwanted components, such as, debris, white blood cells and prostaglandin, are known in the art. For example, swim up, discontinuous (density) gradient and simple (centrifuge) wash.

Numerous commercial temperature controlled modules may be adapted for introducing spermatozoa to an appropriate temperature gradient in accordance with the principles of the present invention. Particularly, up to date automated thermal cyclers designed for robotic Polymerase Chain Reactions (PCRs) may be modified in order to fit the requirements of the present invention

For example, the thermal cyclers of MJ Research (MJR, Waltham, Mass.; e.g. DNA Engine™, Dyad™, Mini-Cycler, PTC-100™, Tetrad™) feature Peltier heating and Alpha™ modules, which are interchangeable heating blocks that allow users to change sample format rapidly. Some of these cyclers feature Hot Bonnet™ heated lids and can be used for a variety of sample formats including microwell plates and even microscope slides.

Another suitable system is the Smart Cycler® instrument (Cepheid, Sunnyvale, Calif.). The system is based on the company's I-CORE® technology-microfluidics-based, temperature-controlled modules that permit each sample to be subjected to different experimental conditions.

Stratagene's RoboCycler (La Jolla, Calif.) offers another suitable temperature controlled modulus. The RoboCycler features four programmable blocks and offers a gradient feature to simplify optimization. This cycler unique is that it employs a robotic arm to move samples from block to block, wherein the temperatures in each block may be distinct.

According to yet another embodiment, the apparatus and system of the invention comprise at least one passage between the at least one first compartment and the at least one second compartment (8). Sperm motility through the passage, between the at least one first compartment and the at least one second compartment, is feasible. The passage may be a film, a membrane or any suitable partition between each first and second distinct compartments. Alternatively, the passage may have the form of a tube extended from each first and second distinct compartments. According to one embodiment the passage may further comprise a matrix between the at least one first compartment and the at least one second compartment, wherein the matrix is selectively permeable to spermatozoa. The matrix may be comprised of any suitable biocompatible material such as a biocompatible gel or collagen among others.

According to yet another embodiment, the apparatus and system further comprising means for monitoring spermatozoa density and motility between the at least one first compartment and the at least one second compartment. According to a preferred embodiment, the means for monitoring sperm motility are operable while the temperature gradient is applied.

Using the system and methods of the present invention enables detection, selection and retrieval of a subpopulation of spermatozoa enriched with capacitated spermatozoa. Spermatozoa enrichment according to the system and methods of the invention comprises exposing a population of spermatozoa to a suitable temperature gradient. The enriched subpopulation may be used for further applications as the systems and methods of the present invention do not impair sperm motility.

Methods known in the art for isolation and detection of capacitated spermatozoa are toxic. Typically, capacitated spermatozoa are identified according to their ability to undergo the acrosome reaction (Bahat et al., ibid). Numerous methods for evaluating acrosome are known in the art. For example, U.S. Pat. Nos. 5,736,346; 5,665,556; and U.S. 5,250,417 among others. The non-toxic morphological detection, often used for evaluating sperm quality, cannot be applied for evaluating spermatozoa capacitation since non-capacitated and capacitated spermatozoa posses essentially a similar morphology. Thus, the present invention confers a major advantage over methods for evaluating and isolating capacitated spermatozoa, since application of the apparatus, systems and methods of the invention enriches a spermatozoa subpopulation for capacitated spermatozoa while not adversely affecting sperm motility.

According to yet another embodiment, prior to exposure of the spermatozoa sample to a temperature gradient, the sample may be incubated with a medium suitable for inducing capacitation. Various media for inducing capacitation in a spermatozoa sample are known in the art. For example, Ham's F-10 (Gibco BRL, life technologies), phosphate-free medium (P-1™, Irvine Scientific, Santa Ana, Calif.) Flushing medium (MediCult, Denmark), Modified HTF medium (Irvine Scientific), Sperm washing medium (Irvine Scientific), Menezo's B2 capacitating medium (Fertility Technologies, Inc., Natick, Mass., USA) and Biggers, Whitten and Whittingham medium, also termed BWW. A typical composition of BWW medium comprises 95 mM NaCl, 4.8 mM KCl, 1.3 mM CaCl₂, 1.2 mM MgSO₄, 1.2 mM KH₂PO₄, 25 mM NaHCO₃, 20 mM sodium lactate, 5 mM glucose, and 0.25 mM sodium pyruvate, pH 7.4 supplemented with Hepes (10 mM, pH 7.4) and BSA (fraction V powder, 3 mg/ml).

According to yet another embodiment, the present invention provides a method for generating a subpopulation of spermatozoa enriched for capacitated spermatozoa, comprising:
(a) providing a population of spermatozoa in at least one first site;
(b) exposing the population of (a) to a temperature gradient induced between the at least one first site and at least one second site, wherein the temperature at the at least one first site is lower than the temperature at the at least one second site;
(c) obtaining a subpopulation of spermatozoa enriched with capacitated spermatozoa from the at least one second site; and, optionally,
(d) repeating step (b) at least once, with the population obtained in (c).

The progression of capacitated spermatozoa in the direction of an increased temperature gradient is commonly termed “thermotaxis” or “thermotactic response”. It is suggested that, in addition to chemotaxis, thermotaxis is a potential cue for guiding spermatozoa to the site of fertilization, wherein chemotaxis describes the response of motile cells or organisms to the gradient of a chemical stimulus, resulting in modulation of the direction of travel so as to approach an attractant or to move away from a repellent. A temperature difference at ovulation, between the sperm reservoir's site (cooler) and the fertilization site (warmer), was found in rabbits, about 2.3° C. between the isthmus and the isthmic-ampullary junction (David, A., et al., Int. J. Gynaec. Obstet., 1972, 10:52-6) and in mated pigs, 0.7° C. between the isthmus and the ampulla (Hunter, R. H. F., Nichol, R. J. Reprod. Fert., 1986, 77: 599-606). Evaluation of thermotaxis in the rabbit spermatozoa is disclosed in Bahat et al., (ibid).

U.S. Pat. No. 5,849,713, by one of the inventors of the present invention, discloses a purified chemotactic factor for human spermatozoa purifiable from human follicular fluid, said factor being of peptidic and of hydrophilic nature causes human spermatozoa concentration-dependent chemotaxis and hyperactivation-like motility.

According to yet another embodiment, step (b) of the method of the invention further comprises monitoring of sperm motility from the at least one first site to the at least one second site. According to yet another embodiment, sperm motility is evaluated in comparison to a standard.

According to yet another embodiment, a subpopulation of spermatozoa enriched with capacitated spermatozoa accumulates in the at least one second compartment of the system and/or apparatus of the invention, upon introduction of a sperm population into the temperature gradient within the culture chamber. Accumulation of the enriched subpopulation in a distinct site, upon utilizing the apparatus, systems or methods of the present invention, is particularly advantageous as it enables a straightforward retrieval of the enriched subpopulation.

Evaluating the percentage of thermotactic spermatozoa may be achieved using kinetic and cell count approaches known in the art. For example, the following kinetic parameters may be measured in a population of spermatozoa exposed to a temperature gradient in accordance with the principles of the present invention:
1. average ΔX—a mean net distance traveled by the spermatozoa during exposure to a temperature gradient. If most, at least more than half, of the spermatozoa in the population travel towards the highest temperature within the temperature gradient, than this value is larger than zero. In the absence of a temperature gradient, this value is approximately zero.
2. % cells with ΔX>0—the percentage of cells which traveled, a net distance, along the temperature gradient towards the highest temperature of the gradient.
3. % cells with ΔX/⊕ΔY|>1—the percentage of cells which traveled a net distance calculated from: % cells with ΔX>0 divided by the percentage of cells which traveled along +ΔY or −ΔY (i.e. |ΔY|), where |ΔY| is perpendicular to the direction of the temperature gradient.

Evaluation of spermatozoa capacitation with respect to a standard may require the construction of a calibration curve by applying the methods and assays of the present invention on a number of different spermatozoa samples having known properties related to their quality. Preferably, the quality of each sample is independently determined by assessing, in portions of each sample, properties such as, morphology, motility, acrosome reactivity, motility and density among others.

The time frame required for exposing a sample of spermatozoa to a temperature gradient in order to enrich the sample with capacitated spermatozoa depends on the components of the system, including the apparatus used, the purpose of the enrichment (e.g. fertility treatment, separation, diagnosis), sperm quality and number of spermatozoa, among others. Evaluating sperm quality using the apparatus, system and methods of the invention, is useful during sperm diagnosis for assessing fertility potential of a sperm population or a semen sample.

A time range for inducing capacitation may range from a few seconds up to the time after which a spermatozoon ceases to be capacitated. Accordingly, exposure of a sample of spermatozoa to a temperature gradient for the purpose of enriching the sample with capacitated spermatozoa may take from less than one hour up to a few hours, typically not exceeding 3-5 hours.

According to yet another embodiment the method and system of the present invention are combined with methods and systems known in the art for obtaining a population of improved spermatozoa. A semen sample may be washed to obtain a population of washed spermatozoa, prior to exposure to a temperature gradient in accordance to the principles of the present invention in order to obtain an improved subpopulation of spermatozoa enriched for capacitated spermatozoa. Semen washing is the process which prepares a semen sample for intrauterine insemination (IUI) among other applications. During spermatozoa washing, a semen sample is washed free of debris, white blood cells, and prostaglandin, which in the case of IUI can cause the uterus to contract. The washing process also removes dead sperm and concentrates the sperm into a small volume. Three main methods of sperm washing are known in the art: swim-up, density gradient wash, and simple wash (centrifugation). The type of wash used depends on the individual characteristics of each semen specimen.

The swim-up is most successful when performed on normal semen and is not recommended for samples of high viscosity, with high numbers of round cells, or with a high content of debris. In this procedure, typically, the washing media is gently placed over the semen in a conical cavity on the bottom of a glass column. The sample is subjected to at least one centrifugation whereas the supernatant is discarded. Medium is gently added to the pellet. Modified spermatozoa washing media (e.g. from Irvine Scientific) may be regularly used to process the sample. Tubes and columns suitable for the swim-up method are commercially available including Zavos Swim-Up Column™ among others. The sample is then placed in an incubator. Incubation time depends on the quality of spermatozoa and is typically within the range of one hour. During this time the spermatozoa are allowed to swim up into the clear media (e.g. Ham's F-10), with the purpose of collecting the most motile, normal spermatozoa, which are free of debris. The isolation media (supernatant) is removed from the swim-up media at the end of incubation, collected and centrifuged, commonly twice, with spermatozoa washing media. The final pellet is then resuspended in a small volume, approximately 0.5 ml. Recovered specimens may be then assessed for various criteria, including: spermatozoa concentration, the percentage and grade of motility, the occurrence of osmotic shock and the percentage of spermatozoa reactive to the hypoosmotic swelling (HOS) test.

The discontinuous (density) gradient method is typically used on samples containing round cells, debris, or those with increased viscosity, but with a relatively normal concentration and motility. The gradient is achieved by layering media of two different densities in a conical tube. The semen is then placed on top of the gradient and the tube is then spun to allow the specimen to proceed through the gradient. The resulting pellet should contain the motile, normal sperm, while the dead spermatozoa and debris are caught up in the gradient media. The pellet is then resuspended in washing media and centrifuged twice. The final pellet is resuspended in a final volume of approximately 0.5 ml of media. Several commercial kits are available for this purpose, e.g. Enhance-S Plus kits of Conception Technologies (San-Diego, Calif.) and the Isolate Sperm Separation Medium of Irvine Scientific (Santa Ana, Calif.).

The simple (centrifuge) wash may be performed on a sample that has a decreased concentration and/or motility. A sample containing round cells and debris is normally not washed by this method. Spermatozoa washing media is added to the specimen and centrifuged. The pellet is recovered, resuspended and again centrifuged. The final pellet is resuspended in a small volume of medium, approximately 0.5 ml.

Application of the apparatus and systems of the present invention has proven particularly effective for monitoring thermotaxis even at very low temperature gradients (about 0.5° C.). The level of thermotactic response detected in low temperature gradients was unexpectedly found essentially similar to the thermotactic response, which was detected in higher temperature gradients, of about 2° C.

According to yet another embodiment, the present invention provides an improvement in a method for inseminating animals or treating human infertility, wherein the improvement comprises applying the method of the invention for selecting and retrieving a subpopulation of spermatozoa enriched for capacitated spermatozoa prior to initiation of the fertility treatments. A major goal of fertilization processes is to increase the ability of spermatozoa to penetrate the egg, i.e. capacitation. Combining the methods, systems and apparatus of the present invention with fertility treatments has the advantage of conducting the fertility treatment with an enriched spermatozoa subpopulation having an increased fraction of capacitated spermatozoa, and thus improved sperm quality. The improvement is achieved by subjecting a population of spermatozoa, prior to the fertility treatment, to a temperature gradient. The resulting enriched subpopulation can be than used for a fertility treatment, such as, artificial insemination, intrauterine insemination (IUI), intracytoplasmic sperm injection (ICSI), in vitro fertilization (IVF), intra-vaginal fertilization, sperm donor insemination and micromanipulation IVF among others.

For intrauterine insemination, the enriched spermatozoa subpopulation is delivered intrauterine in order to initiate a fertility procedure. Specific delivery and application of a treatment medium such as spermatozoa, to an intrauterine locus, particularly for the successful insemination of an egg thereat, is an intended yet illusive goal of many in the medical field. The likelihood of such fertilization occurs, by the successful delivery of the spermatozoa and its association with the extracellular coating of the oval called the zone pellucid. Once a successful motile spermatozoon has fused with the egg membrane, fertilization has been completed. For this to occur, however, millions of spermatozoa must be successfully released so that one of them reaches the egg at the optimum time. This time window for such spermatozoa within the uterus, from introduction to fertilization, may extend in range over a twenty-four to a sixty hour period.

U.S. Pat. No. 6,004,260 discloses a method for the application of a treatment medium to the intrauterine cavity of a female, comprising the steps of: introducing a first pressurizable chamber within a intrauterine cavity of a female; introducing a second pressurizable chamber radially outwardly of said first pressurizable chamber; filling said first pressurizable chamber with a treatment medium; and filling said outer pressurizable chamber with a pressurizable fluid, so as to effect discharge of said treatment medium from said innermost first pressurizable chamber over an extended period of time.

Another approach for delivering material into the female uterus is shown in U.S. Pat. No. 4,182,328 to Bolduc et al. This patent shows a dispensing instrument utilizing a balloon which is inflated within the uterus. A piston and cylinder arrangement has a duct that extends through the balloon, which feeds the material to the uterus. The material is delivered over a short period of time and the balloon and probe are readily withdrawn thereafter. A further concept to Bolduc, is shown in U.S. Pat. No. 4,547,188 with a complicated housing and injector assembly with a conduit path through a balloon for treatment of a female uterus.

U.S. Pat. No. 4,654,025 to Cassou et al, discloses an insemination apparatus for animals, utilizing a flexible injector probe, having a plurality of expandable balloons one of each end thereof to facilitate injection of semen from a reservoir tube into the vaginal cavity of the animal. U.S. Pat. No. 5,104,377 to Levine et al, shows a device for accessing and introducing fluids into the female uterus. This device uses several spaced-apart balloons to securely couple the shaft to the uterus, adjusting to the length of the cervical canal. U.S. Pat. No. 5,372,584 to Zink et al, shows an apparatus for establishing access to the uterus and fallopian tubes of a female. An anchoring tube on the end of a flexible catheter is first inserted within the uterus. After such anchoring is completed, the elongated second catheter is arranged to extend through the first catheter and balloon and into the fallopian tubes. Injection of treatment into those fallopian tubes is thereby accomplished.

U.S. Pat. No. 5,562,654 to Smith et al. shows an arrangement for time-released delivery of a preparation into a uterine cavity. An osmotic pump is placed within the vagina of the female, having a delivery tube extending within the uterus. An anchoring balloon is disposed about the delivery tube within the uterus and is pressurized through a port, which is pressurized through the vagina. Osmotic pressure gradually builds up within the osmotic chamber to pressurize an inner chamber to deliver material from the vagina to within the uterus through the delivery tube.

### EXAMPLES

### Materials and Methods

Spermatozoa isolation. Rabbit semen was collected with an artificial vagina (IMV technologies, France) and washed twice by centrifugation (120×g, 10 min) with enriched Biggers, Whitten, and Whittingham medium (BWW: 95 mM NaCl, 4.8 mM KCl, 1.3 mM CaCl₂, 1.2 mM MgSO₄, 1.2 mM KH₂PO₄, 20 mM sodium lactate, 5 mM glucose, 0.25 mM sodium pyruvate, 25 mM NaHCO₃, pH 7.4 supplemented with 80 mM Hepes (pH 7.4) and 40 mg/ml BSA). Each spermatozoa sample was analyzed for motility parameters using a Makler counting chamber (Sefi Medical Instruments Ltd., Haifa, Israel) and a computerized spermatozoa analysis program (Hobson Tracking System Ltd., Sheffield, England). The spermatozoa concentration was then adjusted to 10×10⁶ cells/ml and incubated for 14-18 h at 37° C. under an atmosphere of 5% CO₂ to induce capacitation. Human spermatozoa were collected, washed and resuspended in BWW as described by Jaiswal et al. (Biol. Reprod. 60: 1314-19, 1999).

Induction of acrosome reaction and evaluation of response. Capacitated spermatozoa were identified according to their ability to undergo the acrosome reaction. For stimulation, rabbit spermatozoa (2×10⁷ cells/ml) were incubated with A23187 (Sigma; 10 μM from a stock solution in DMSO; the final DMSO concentration was 0.2%) or DMSO (control) for 30 min at 37° C. The acrosome marker Pisum sativum agglutinin labeled with fluorescein isothiocyanate (FITC-PSA, Sigma) was used to visualize the state of the acrosome, using a modification of previously reported staining method. Briefly, after induction with A23187, the samples were fixed with 2% (v/v) formaldehyde for 20 minutes at room temperature, washed twice in PBS at 300×g for 3 minutes, and resuspended in PBS. An aliquot was put on the slide, dried, and permeabilized in methanol for 2 minutes at room temperature. After washing in double-distilled water and drying in air, 50 μg/ml PSA-FITC were added for 15 minutes at room temperature in the dark. After washing and drying, the slides were mounted with elvanol and the coverslips were sealed with acrylic nail polish. The samples were observed under an inverted fluorescence microscope (Nikon Te300).

Thermotaxis Assay. The assays were carried out in a modified Zigmond chamber (FIG. 1), consisting of two elliptic wells of 1 ml, separated by a partition wall of 1 mm in width. The chamber was closed from above with a coverslip and sealed with hot wax, leaving a space at the order of 10 μm between the coverslip and the partition wall. Each well was filled with a spermatozoa suspension (2×10⁶ motile cells/ml) through a small hole at the side of the well, after which the hole was sealed with hot wax. The temperature was controlled in each well by two tubes connected to a temperature controlled water bath. One bath was at a temperature T₁ and the other at T₂. In control experiments with no temperature gradient, the tubes of both wells were connected to a single bath, either at T₁ or at T₂. The temperature at each well was directly measured by a thermocouple connected to a digital thermometer (±0.2° C. accuracy). The movement of cells on top of the partition wall, in the middle of the field between the two wells, was video recorded for 15 min following the sealing of the chamber. The tracks made by the spermatozoa during the last 5 min of each recording were analyzed by a computerized motion analysis system (Hobson Sperm Tracker System Ltd., Sheffield, England).

### Example 1

### Temperature Measurement at the Oviduct of a Rabbit

Temperatures within the oviduct were measured using two thermistor probes, 0.5 mm in width and 30 cm in length, connected to two digital thermometers (±0.2° C. accuracy). Temperature was measured under anesthesia at three sites: the isthmus (near the uteri-isthmic junction), the isthmic-ampullary junction, and, as a control for body temperature, the rectum. The measurement at the rectum was required in order to evaluate temperature loss due to anesthesia and due to the open abdomen. All measurements were carried out at ovulation (10.5-11.0 h post-mating). The temperatures in the isthmus and the isthmic-ampullary junction were as follows: 3.1±0.4° C. and −1.5±0.8° C. (mean±SD of 4 oviducts), respectively, relative to the rectal temperature. These results confirmed the published ˜2° C. difference between the storage and fertilization sites and further suggested that this difference is achieved by a reduced temperature at the spermatozoa reservoir's site rather than by an elevated temperature at the fertilization site.

### Example 2

### Thermotactic Response Measurements in Capacitated Spermatozoa

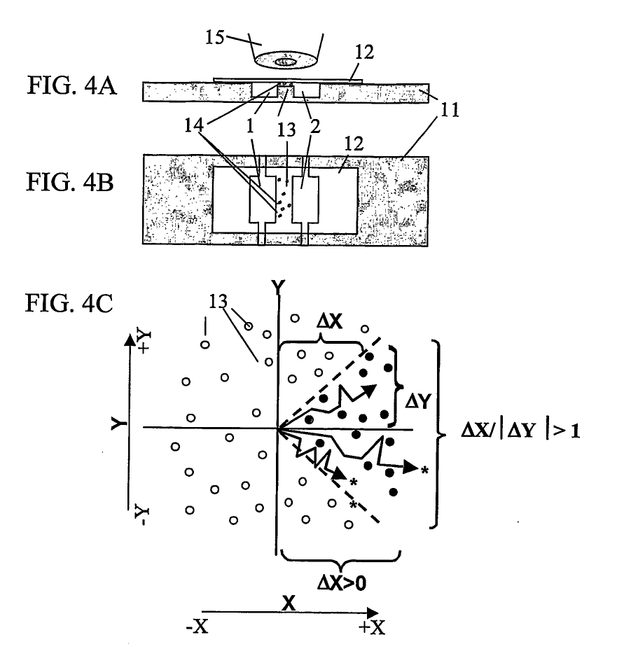

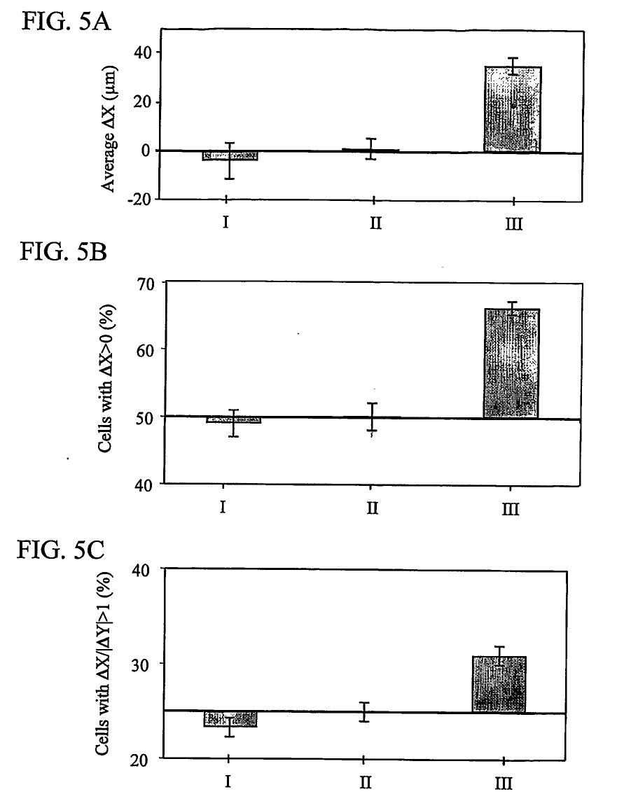

A directionality-based assay, independent of the spermatozoa's speed and pattern of movement for the measurement of thermotaxis was employed (Fabro et al., Biol. Reprod. 2002, 67:1565-71). For this purpose a modified Zigmond chamber consisting of two parallel wells separated by a partition wall was used (FIG. 1 and FIG. 4; Bahat et al., ibid). The temperature in each well could be accurately controlled and measured (±0.2° C.). An equal concentrations of rabbit spermatozoa (pre-incubated for 14-18 hours under capacitating conditions) was added to each well and the tracks of the spermatozoa movement on top of the partition wall was monitored and recorded. Thermotaxis was then analyzed using a computerized motion analysis system, based on three directionality parameters: a mean net distance traveled along the temperature gradient (termed: average ΔX), percentage of cells whose net distance of traveling was towards the warmer well (termed: cells with ΔX>0) and percentage of cells traveling a longer distance in the direction of the temperature gradient than in the direction of no-gradient (|ΔY|, which is perpendicular to ΔX (termed: cells with ΔX/|ΔY|>1). The temperature in each well was either different, or similar. In the first case, the temperature difference was of 2° C. between the wells, which is approximately the temperature difference at the oviduct during ovulation between the spermatozoa reservoir and the fertilization site. The second case served as a control for the temperature gradient, whereas the temperature in both well was maintained at either 37° C. or at 39° C. As both wells were at the same temperature, all three directionality parameters had values expected for random movement, that is ˜0 μm for average ΔX, ˜50% for the percentage of cells with ΔX>0 and ˜25% for the percentage of cells with ΔX/|ΔY|>1. However, under a temperature gradient of about 2° C. difference between the wells, all the directionality parameters were larger than the expected values for a random movement (FIG. 5; see also Bahat et al., ibid), indicating the occurrence of spermatozoa thermotaxis under a temperature gradient of 37° C.-39° C. In other words, the spermatozoa can navigate in accordance with a temperature gradient.

The speed of the spermatozoa movement was evaluated using the following kinetic parameters (Table 1):
1. VCL-curvilinear velocity, the time-average velocity of the spermatozoa head along its actual trajectory;
2. VSL-straight line velocity, also termed progressive velocity which is the time-average velocity of the spermatozoa head along a straight line from its first position to its last position;
3. LIN-percent linearity that is the ratio VSL/VCL multiplied by 100;
4. STR-percent straightness calculated as the ratio between the straight line from the first point on the smoothed path to the last point on this path and the total distance along the smoothed path, multiplied by 100;
5. MOT-percent motile cells;
6. HYP-percent hyperactivated cells that is cells having a motility pattern characterized by increased velocity, decreased linearity, increased amplitude of lateral head displacement, and flagellar whiplash movement.

It was found that the speed and motion pattern were not significantly affected by the temperature difference (Table 1; see also Bahat et al., ibid). Furthermore, the values of the kinetic parameters under a temperature gradient (37° C./39° C.) were not significantly different from the respective control values, as determined by ANOVA Repeated Measures Analysis of Variance.

TABLE 1

| Kinetic | Control | Control | Temp. Gradient |
|---|---|---|---|
| parameter* | (37° C./37° C.) | (39° C./39° C.) | 37° C./39° C. |
| VCL (μm/s) | 110 ± 5 | 104 ± 6 | 111 ± 2 |
| VSL (μm/s) | 58 ± 3 | 59 ± 5 | 55 ± 4 |
| LIN (%) | 51 ± 2 | 54 ± 2 | 49 ± 3 |
| STR (%) | 83 ± 2 | 85 ± 1 | 78 ± 3 |
| MOT (%) | 83 ± 9 | 87 ± 8 | 97 ± 2 |
| HYP (%) | 6 ± 2 | 4 ± 2 | 14 ± 3 |

Without wishing to be bound by any mechanism, this finding together with the probability that spermatozoa chemotaxis is restricted to the immediate surroundings of the egg suggest that spermatozoa thermotaxis and chemotaxis are long- and short-range mechanisms, respectively, which occur consecutively whereas each of these processes occurs within a region that is not functional for the other process. In other words, each one is functional in a different region, where the other mechanism is ineffective. One region is between the isthmic spermatozoa reservoir and the fertilization site, and the other is in the immediate vicinity of the egg, especially within the viscous milieu of the cumulus oophorus that surrounds the egg. The course of action is initiated when capacitated spermatozoa are released from the isthmic spermatozoa reservoir, and are presumably guided by thermotaxis towards the warmer fertilization site. Then, at close proximity to the egg and within the cumulus mass, spermatozoa guidance is carried out by chemotaxis. It is believed that each of these mechanisms is essential and cannot be replaced by the other mechanism. Spermatozoa chemotaxis apparently cannot occur in the first region because of peristaltic movements of the oviduct that presumably prevent the formation of a long-range chemoattractant gradient. Since these movements are not expected to affect a temperature gradient, it is reasonable that only thermotaxis can be functional in this region. In the other region, which is in the immediate vicinity of the egg and within the cumulus, the opposite seems to hold. In that region a measurable temperature gradient probably cannot be maintained, whereas a chemoattractant gradient seems very effective because of the relatively short distances and the viscoelastic milieu of the cumulus that resists the stirring action of the oviduct. In spite of the relatively short distance, spermatozoa chemotaxis seems essential in this region. Without it, it is difficult to rationalize how the first few spermatozoa that enter the cumulus find the egg so effectively. The observation that only capacitated spermatozoa can penetrate the cumulus is consistent with this notion.

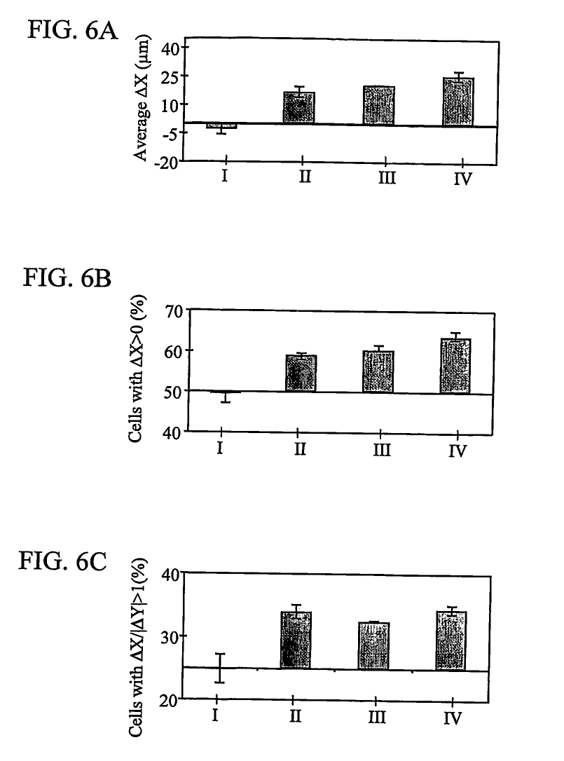

It should be noted that the distance between the spermatozoa reservoir and the fertilization site in the rabbit female genital tract is larger than the 1 mm, which was the distance between the wells of the modified Zigmond chamber. Accordingly, the temperature gradient sensed by the spermatozoa in vivo is probably shallower. In order to examine spermatozoa responsiveness to shallower temperature gradients, the temperature difference between the wells of the modified Zigmond chamber was reduced into a gradient of 1° C. and 0.5° C. The strong thermotactic response repeated itself, in a similar manner to the response detected under a temperature gradient of 2° C. (FIG. 6; see also Bahat et al., ibid). These results verifies that spermatozoa can sense and respond to shallow temperature gradients.

### Example 3

### Thermotactic Response Measurements in Non-Capacitated Spermatozoa

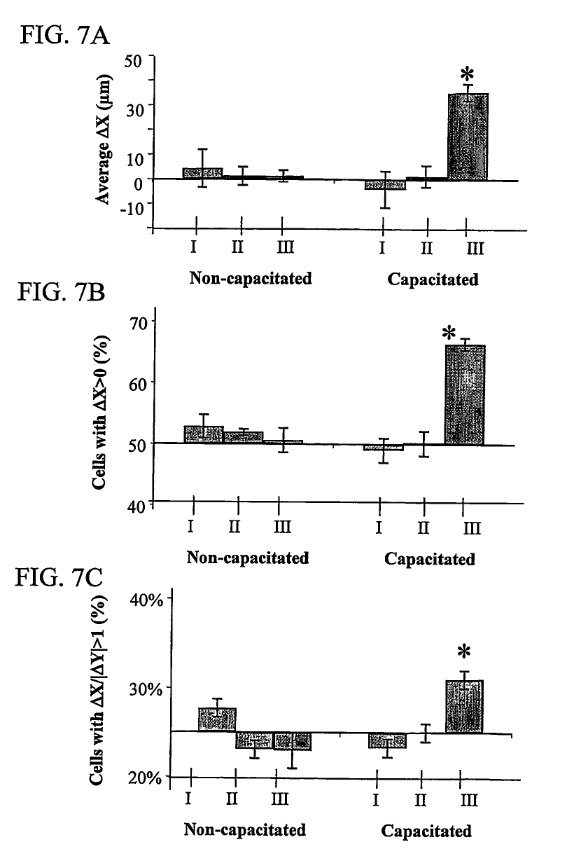

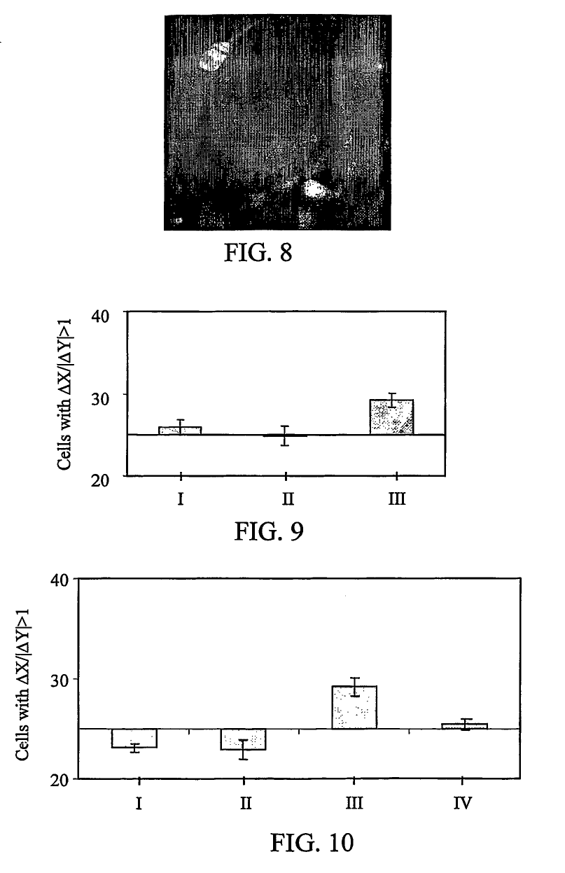

In the above example it was demonstrated that only a fraction of the spermatozoa are thermotactically responsive, as is the situation in sperm chemotaxis, where only a fraction of the sperm-population, the fraction of capacitated cells is responsive to the chemoattractant gradient. To determine whether only capacitated cells are thermotactically responsive the thermotactic responsiveness of spermatozoa was studied, 1 h post-ejaculation. The thermotactic responsiveness was measured in spermatozoa that did not undergo 14-18 h pre-incubation (rabbit spermatozoa become capacitated only after a long delay of about 10 hours of incubation under capacitating conditions). Under these conditions, no capacitated cells were detected as compared to 15.7±2.6% (±SEM) capacitated cells in spermatozoa that were pre-incubated under capacitating conditions for 16 h. The level of capacitated spermatozoa was determined from the difference in the levels of acrosome-reacted cells prior to and after the induction of the acrosome reaction with A23187 (FIG. 8; see also Bahat et al., ibid). Using the acrosome reaction assay, there was essentially no difference in all three parameters for thermotaxis, between spermatozoa in the temperature gradient and spermatozoa in the no-gradient controls. The results suggest that thermotactic responsiveness is acquired during sperm capacitation, as does chemotactic responsiveness. It was further observed that when spermatozoa was exposed to a temperature gradient of 2° C., starting with incubation in the 37° C. well and progression towards the 39° C. well, the level of capacitated spermatozoa at the 39° C. well was about two-fold higher than that in the original 37° C. The observations that the thermotactic response appeared to be restricted to capacitated spermatozoa and that only 15.7±2.6% of the spermatozoa were capacitated may explain the relatively small fraction of thermotactically responsive cells (7-17% according to FIG. 7).

### Example 4

### Thermotactic Response in Human Spermatozoa

Thermotaxis was examined in capacitated human spermatozoa assuming that a temperature difference also exists at ovulation in the Fallopian tube of humans. Capacitation was achieved by incubating the spermatozoa for 2 h in BWW medium. The medium was supplemented with 0.3% BSA in order to obtain maximal level of capacitated cells.

A temperature difference of 2° C. between two wells of a modified Zigmond chamber (FIGS. 1 and 4), similar to the difference within the rabbit oviduct at ovulation, was applied where the temperature of the first well was 37° C. and of the second well was 39° C. For non-gradient controls the same temperature of either 37° C. or 39° C., was applied in the two wells. In the absence of a temperature gradient the percentage of cells with ΔX/|ΔY⊕>1 was 25% (FIG. 9, I and II) and in the presence of a temperature gradient this parameter was 29.5%, which is a higher percentage than the expected values for a random movement (FIG. 9, III). The results, given with respect to the expected values in the case of random movement, are average of nine determinations±S.E.M. The total numbers of cells analyzed were 11,533-13,109. Moreover, the differences between the results in the presence of a 37° C./39° C. gradient versus the controls (absence of a temperature gradient) were extremely significant, p<0.0001).

The results clearly suggest the occurrence of sperm thermotaxis. The result further demonstrated that only a fraction of the human spermatozoa, about 3.0 to 5.2%, were thermotactically responsive. It may be suggested that this thermotaxis responsive fraction of the sperm population is the fraction of capacitated cells within the population as is the case with chemotaxis and responsiveness to the chemoattractant gradient (Cohen-Dayag, ibid.). It was further found that the percentage of responsive cells was lower than the corresponding value in rabbit spermatozoa. However, the level of capacitated cells is lower in human spermatozoa than in rabbit spermatozoa.

Thermotactic response of capacitated versus non-capacitated human spermatozoa was analyzed. A non-capacitated population of spermatozoa was obtained by washing and re-suspending a spermatozoa sample in a non-capacitating medium (NCM), which is devoid of BSA, bicarbonate and calcium ions. Spermatozoa incubation with the NCM medium resulted in a low level of capacitated cells which was 2.5 time fold lower than the level of capacitated cells in a sample incubated with a BWW medium. The results were evaluated with respect to the expected values in the case of random movement and are the average of nine determinations±S.E.M. The total numbers of cells that were analyzed is about 6180-7839. The differences between capacitated cells under a 37° C./39° C. gradient (FIG. 10, III) and control (FIG. 10, I and II) or non-capacitated cells under a 37° C./39° C. gradient (FIG. 10, IV) were highly significant, p<0.0001.

The thermotactic response of cells that were exposed to a temperature gradient but were not pre-incubated with NCM (FIG. 10, IV) was essentially similar to that obtained from spermatozoa incubated with NCM following incubation in a constant temperate (no gradient controls; FIG. 10, I and II).

### Example 5

### In Vitro Fertilization (IVF) and IUI Treatments—Clinical Study

A prospective, double blind, controlled randomized trial assesses the efficacy of using spermatozoa enriched with capacitated cells according to the principles of the present invention. Analysis is performed following enrolment of patients undergoing fertility treatments. A major emphasis is directed toward guaranteeing that the correct spermatozoa is used with the correct eggs through precise labeling and confirmation systems.

Semen Preparation for Oocyte Insemination. Semen samples are analyzed using a suitable automated device, e.g. CellSoft automated semen analyzer (CRYO Resources Ltd., NY, USA). Samples are analyzed by selection criteria including motion analysis, average sperm density, average motility and average normal morphology according to the World Health Organization (WHO). Suitable spermatozoa samples are selected and maintained at 37° C. for 15 to 20 minutes prior to insemination. For all samples, swim-up preparation of the spermatozoa is carried out in aliquots, of about 0.5 ml which are randomly divided into two groups one group is marked “non-enriched” and the other group is marked “enriched”. The aliquots of both groups are topped with 0.5 ml culture media, consisting of Whittingham T6 plus 15% cord serum, and incubated for 60 minutes. The supernatant is pooled into a sterile 5 ml tube and spun at 900×g for 10 minutes. The supernatant above the pellet is then discarded, and the pellet resuspended in a 0.2 ml culture medium. Aliquots are immediately analyzed and labeled as “post swim up” (POS). The samples of the “non-enriched” group are placed in the incubator at 37° C. until insemination time, approximately five hours later. The samples of the “enriched group” are exposed to a temperature gradient using the systems and methods of the present invention until insemination time.

Patients. Women who signed informed consent and who fulfill inclusion criteria are randomized in a 1:1 ratio to receive the IVF or IUI treatments with either a regular spermatozoa sample or with an enriched spermatozoa sample. All women typically receive hormonal ovarian stimulation, including follicle stimulating hormones such as pergonal, gonal-F, and clomiphene citrate, at various doses depending on patient's clinical parameters. hCG (at least 5,000 IU) is administered when at least one lead follicle is 18-20 mm. Oocyte aspiration or sperm insemination for IUI are carried out approximately 34 hr after hCG administration. For IUI treatment, Kremer Delafontaine catheter is used.

In Vitro Fertilization. About six hours after oocyte(s) aspiration, the oocyte(s) (initially divided into two groups labeled “normal” or “enriched”) are inseminated by adding approximately 50 to 500×10³ of regular or enriched spermatozoa, depending on the subjective judgment of the embryologist, to each dish containing up to four oocytes. Inseminated oocytes are checked for fertilization 18 hours later. Normal fertilization is defined by the presence of gamete fusion sequel.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-51. (canceled)

52. An apparatus for selecting a subpopulation of spermatozoa, comprising:
(a) a culture chamber having at least one first compartment, at least one second compartment and a passage enabling spermatozoa access between the at least one first compartment and the at least one second compartment; and,
(b) means for generating a discrete or continuous temperature gradient between the at least one first compartment and the at least one second compartment such that the temperature in said at least one first compartment is lower than the temperature in said at least one second compartment.

53. The apparatus according to claim 52, wherein the culture chamber is adapted for containing culture medium suitable for maintaining the motility of mammalian spermatozoa.

54. The apparatus according to claim 52, the passage further comprising a matrix between the at least one first compartment and the at least one second compartment.

55. The apparatus according to claim 54, wherein the matrix is selectively permeable to spermatozoa.

56. The apparatus according to claim 55, wherein the matrix comprises a material selected from the group consisting of: a biocompatible gel, fibrin substrate, silicon, carbon blocks or fibers, polysaccharides and collagen.

57. The apparatus according to claim 52, wherein the culture chamber comprises a biocompatible material selected from the group consisting of: glass, polycarbonate, polyethylene, polyurethane, ethylene-vinylacetate copolymer and polyolefins.

58. The apparatus according to claim 52, wherein the culture chamber is sterile or aseptic.

59. The apparatus according to claim 52, further comprising means for monitoring sperm motility.

60. The apparatus according to claim 52, wherein the culture chamber is disposable.

61. The apparatus according to claim 52, wherein the temperatures within the temperature gradient are suitable for maintaining sperm viability and the difference between the highest and the lowest temperatures of the temperature gradient is between 0.05° C. to 20° C.

62. A system for generating a subpopulation of spermatozoa enriched for capacitated spermatozoa, comprising the apparatus of claim 52 and further comprising means for retrieving spermatozoa from said at least one second compartment.

63. The system according to claim 62, further adapted for employing semen washing.

64. A method for generating a subpopulation of spermatozoa enriched with capacitated spermatozoa, comprising:
(a) providing a population of spermatozoa in at least one first site;
(b) exposing the population of (a) to a discrete or continuous temperature gradient induced between the at least one first site and at least one second site, wherein the temperature at the at least one first site is lower than the temperature at the at least one second site;
(c) obtaining a subpopulation of spermatozoa enriched with capacitated spermatozoa from the at least one second site; and, optionally,
(d) repeating step (b) at least once, with the population obtained in (c).

65. The method according to claim 64, wherein step (b) further comprises monitoring sperm motility from the at least one first site to the at least one second site.

66. The method according to claim 64, wherein the population of spermatozoa comprises non-human mammalian spermatozoa.

67. The method according to claim 64, wherein the population of spermatozoa comprises human spermatozoa.

68. The method according to claim 64, wherein the temperatures within the temperature gradient are suitable for maintaining sperm viability and the difference between the highest and the lowest temperatures of the temperature gradient is no between 0.05° C. to 20° C.

69. The method according to claim 64, further comprising retrieving a population of spermatozoa after step (b) from the at least one second site.

70. The method according to claim 69, wherein the retrieved spermatozoa is utilized for a fertility treatment.

71. The method according to claim 70, wherein the fertility treatment is selected from the group consisting of: artificial insemination, intrauterine insemination (IUI), intracytoplasmic sperm injection (ICSI), in vitro fertilization (IVF), micromanipulation IVF and intra-vaginal fertilization.

72. The method according to claim 64, further comprising semen washing prior to step (a).

73. An assay for evaluating sperm quality in a population of spermatozoa, comprising:
(a) providing a population of spermatozoa in a first site;
(b) exposing the population of (a) to a discrete or continuous temperature gradient induced between the first site and at least one second site, such that the temperature at the at least one second site is higher than at said first site; and,
(c) evaluating the percentage of spermatozoa within the population accumulated at the second site of (b) in comparison to a standard sperm population, wherein the percentage of spermatozoa migrating along the temperature gradient between said first site and the at least one second site is a measure of sperm quality.

74. The assay according to claim 73, wherein the population of spermatozoa comprises mammalian spermatozoa.

75. The assay according to claim 74, wherein the population of spermatozoa comprises human spermatozoa.

76. The assay according to claim 73, wherein the temperatures within the temperature gradient are suitable for maintaining sperm viability and the difference between the highest and the lowest temperatures of the temperature gradient is between 0.05° C. to 20° C.
